(19) United States
(12) Patent Application Publication
(10) Pub. No.: US 2017/0347938 A1
(43) Pub. Date: Dec. 7, 2017

(54) METHOD AND APPARATUS FOR USE IN ALLERGY TESTING

(71) Applicant: Koninklijke Philips N.V., Eindhoven (KR)

(72) Inventors: Maria Estrella MENA BENITO, Eindhoven (NL); Ihor Olehovych KIRENKO, Veldhoven (NL)

(21) Appl. No.: 15/536,670

(22) PCT Filed: Dec. 10, 2015
(86) PCT No.: PCT/EP2015/079217

§ 371 Date: Jun. 16, 2017

(30) Foreign Application Priority Data
Dec. 18, 2014 (EP) 14198854.3

(51) Int. Cl. A61B 5/00; A61B 5/024; A61B 10/00; A61B 90/00

(52) U.S. Cl. CPC A61B 5/411; A61B 10/0035; A61B 5/02433; A61B 5/7282; A61B 2090/3937; A61B 2562/0233; A61B 2576/00

(57) ABSTRACT

There is provided a method for use in determining whether a subject is allergic to a substance. The method comprises: receiving a first set of spatially distributed light intensity values covering a skin region of the subject including a location at which the substance has been applied; wherein the light intensity values in the first set are intensities of visible light; receiving a second set of spatially distributed light intensity values covering the skin region, wherein the light intensity values in the second set are intensities of infrared, IR, light; generating a first spatial distribution of photoplethysmogram, PPG, pulse amplitudes based on the first set of light intensity values; generating a second spatial distribution of PPG pulse amplitudes based on the second set of light intensity values; comparing the first spatial distribution to the second spatial distribution, and to the location at which the substance has been applied; and outputting an indication of whether the subject is experiencing an allergic reaction to the substance based on the comparing.

### TECHNICAL FIELD OF THE INVENTION

The invention relates to a method and apparatus for use in determining whether a subject is allergic to a given substance.

### BACKGROUND TO THE INVENTION

An allergy is a hypersensitivity disorder of the immune system. Symptoms include red eyes, itchiness, runny nose, eczema, hives, or an asthma attack. Allergic reactions occur when a person's immune system reacts to normally harmless substances in the environment. A substance that causes an allergic reaction is called an allergen. Effective management of an allergy, e.g. by avoidance and environmental control measures, relies on an accurate diagnosis of the allergen responsible for the symptoms experienced by a subject. A variety of tests exist to diagnose allergic conditions.

Skin testing is one of the most sensitive ways to identify a substance or substances that are causing allergy symptoms. One such test is the skin prick test, which is commonly used to diagnose allergies to house dust mites, grass pollens and cat dander. The test involves marking areas of the skin with a pen to identify each allergen that will be tested. A drop of extract for each potential allergen is placed on the corresponding mark, and then the skin is pricked so the extract can enter into the outer layer (epidermis) of the skin. The reaction of the skin is evaluated after 30 minutes. A disadvantage of the skin prick test is that interpretation of the results is difficult in patients with eczema or dermatographism.

Another type of allergy skin test is the intracutaneous test. This test involves injecting a small amount of allergen into the skin using a hypodermic needle. The intracutaneous test can only be performed by allergy specialists in specialist centres, and is thus used relatively rarely.

The patch test is widely used for diagnosing contact allergic dermatitis. Patch testing can identify whether a substance that comes in contact with the skin is causing inflammation of the skin. Possible allergens are applied in a standardized form (e.g. an adhesive patch which is divided into multiple separate spatial areas, each of which contains a different allergen) to a healthy area of the patient's skin. The patch is left in place for 48 hours, and the various potential allergens are thereby held against the subject's skin continuously during this time. A permanent or surgical marker is used to mark the location of the patch on the subject's skin, so that the test area can be re-examined when the patch is no longer present. Typically an initial reading of the test is performed as soon as the patch is removed, and then an additional reading is made 3 to 4 days after the initial placement (i.e. 1-2 days after removal).

The patch test can be performed either with the suspected chemicals or with the standard series of allergens. FIG. 1a shows a test patch 10 in use on a subject. The test patch 10 comprises ten substance containing regions 11, each of which contains a different potential allergen. FIG. 1b shows the subject's skin immediately after the patch 10 has been removed. It can be seen that a reaction 12 in the form of a red skin weal was caused by one of the substances contained in the patch 10.

The classification and score grading of patch test reactions depends on descriptive morphology. Typical morphological features of an allergic (i.e. positive) test response are erythema (redness), oedema (swelling), papules (solid bumps) and vesicles (fluid-filled bumps). An erythematous infiltration and/or papules must both occur for a reaction to be considered allergic. By contrast, reactions that show only erythema without infiltration (known as doubtful reactions) are frequently non-specific and/or are caused by irritation rather than an allergy. The size of the weal/irritated area is also taken into consideration. Allergic patch test reactions are traditionally scored in terms of intensity, using a grading scale from 1+ to 3+ (with 1+ corresponding to a weak positive reaction and 3+ corresponding to an extreme positive reaction). FIG. 2 shows examples of patch test reactions. The reactions shown can be classified as (clockwise from the top left): negative; negative (an irritant reaction); doubtful; 1+ positive; 2+ positive; and 3+ positive. Sometimes the results can be inconclusive or misleading. For example, in some cases instead of one or two positive reactions, sometimes nearly all test areas become red and itchy (thus generating a false positive result). This is known as ‘angry back’ and is most likely to occur in subjects who have very active dermatitis. In other cases, there may be little or no apparent reaction to a substance that regularly causes dermatitis in that person (a false negative result). It will be appreciated that distinguishing between allergic and irritant reactions is of major importance in the interpretation of patch tests.

The reading of a patch test reaction is subjective, based on inspection and palpation of the test responses by a medical professional. An allergist therefore needs to be thoroughly trained in order to be able to reliably interpret the results of patch testing. In practice there exists considerable inter-individual variation in how patch tests are both read and then interpreted by clinicians. The allergist's background knowledge and experience can greatly affect the results. For instance, while some allergists might evaluate a homogeneous redness in just part of the test area as 1, for others a score of 1 implies a homogeneous redness in the whole test area.

A further disadvantage of the patch test is that the patch must be kept dry whilst it is attached to the subject. This means that only a sponge batch can be taken, and that excessive sweating should be avoided. Also, the patches should not be exposed to sunlight or other sources of ultraviolet (UV) light. Patch testing can therefore cause significant disruption to a subject's daily routine, for several days.

There is therefore a need for a fast, convenient and reliable way to determine whether a subject is allergic to a given substance.

### SUMMARY OF THE INVENTION

According to a first aspect of the invention, there is provided a method for use in determining whether a subject is allergic to a substance. The method comprises:
receiving a first set of spatially distributed light intensity values covering a skin region of the subject including a location at which the substance has been applied; wherein the light intensity values in the first set are intensities of visible light;
receiving a second set of spatially distributed light intensity values covering the skin region, wherein the light intensity values in the second set are intensities of infrared, IR, light;
generating a first spatial distribution of photoplethysmogram, PPG, pulse amplitudes based on the first set of light intensity values;
generating a second spatial distribution of PPG pulse amplitudes based on the second set of light intensity values;
comparing the first spatial distribution to the second spatial distribution, and to the location at which the substance has been applied; and
outputting an indication of whether the subject is experiencing an allergic reaction to the substance based on the comparing.

Thus, systems according to the invention enable a skin-patch based allergy test which is both objective and quantitative, whilst also being more sensitive and more specific than conventional skin patch-based allergy tests. A further advantage is that systems according to the invention can provide a result much more quickly than conventional tests, and therefore cause considerably less inconvenience to the subject.

In some embodiments the first set of spatially distributed light intensity values are obtained simultaneously with the second set of spatially distributed light intensity values. In other embodiments the first set of spatially distributed light intensity values and the second set of spatially distributed light intensity values are obtained sequentially.

In some embodiments comparing the first spatial distribution to the second spatial distribution comprises identifying regions of high pulse amplitude in the first spatial distribution by comparing the pulse amplitudes in the first spatial distribution to a first threshold; identifying regions of high pulse amplitude in the second spatial distribution by comparing the pulse amplitudes in the second spatial distribution to a second threshold; and comparing the identified regions of high pulse amplitude in the first spatial distribution to the identified regions of high pulse amplitude in the second spatial distribution. In some embodiments the second threshold is the same as the first threshold. In alternative embodiments the second threshold is different to the first threshold.

In some embodiments the method further comprises:
receiving a first baseline set of spatially distributed light intensity values covering the skin region, wherein the light intensity values in the first baseline set are intensities of visible light;
receiving a second baseline set of spatially distributed light intensity values covering the skin region, wherein the light intensity values in the second baseline set are intensities of infrared, IR, light;
generating a first baseline spatial distribution of PPG pulse amplitudes based on the first baseline set of light intensity values; and
generating a second baseline spatial distribution of PPG pulse amplitudes based on the second baseline set of light intensity values.

In some embodiments the first baseline set of spatially distributed light intensity values are obtained simultaneously with the second baseline set of spatially distributed light intensity values. In other embodiments the first baseline set of spatially distributed light intensity values and the second baseline set of spatially distributed light intensity values are obtained sequentially.

In such embodiments the light intensity values in the first and second baseline sets were obtained before the substance was applied to the skin region.

In some such embodiments the method further comprises:
subtracting the first baseline spatial distribution of PPG pulse amplitudes from the first spatial distribution of PPG pulse amplitudes to generate a corrected first spatial distribution of PPG pulse amplitudes; and
subtracting the second baseline spatial distribution of PPG pulse amplitudes from the second spatial distribution of PPG pulse amplitudes to generate a corrected second spatial distribution of PPG pulse amplitudes.

In such embodiments comparing the first spatial distribution to the second spatial distribution, and to the location at which the substance has been applied comprises comparing the corrected first spatial distribution to the corrected second spatial distribution, and to the location at which the substance has been applied.

In some embodiments the method further comprises:
identifying regions of high pulse amplitude in the first baseline spatial distribution by comparing the pulse amplitudes in the first baseline spatial distribution to a threshold;
identifying regions of high pulse amplitude in the second baseline spatial distribution by comparing the pulse amplitudes in the second baseline spatial distribution to a threshold; and
selecting a skin location for application of the substance based on the identified regions of high pulse amplitude in the first and second baseline spatial distributions.

In some embodiments selecting a skin location for application of the substance comprises selecting a skin location which is not within any of the identified regions of high pulse amplitude. In some embodiments the method further comprises applying a mark to the subject's skin at the selected location.

In some embodiments the first set of spatially distributed light intensity values comprises spatially distributed light intensity values covering the skin region obtained at a first time, and spatially distributed light intensity values covering the skin region obtained at a second, later, time. In such embodiments the second set of spatially distributed light intensity values comprises spatially distributed light intensity values covering the skin region obtained at the first time, and spatially distributed light intensity values covering the skin region obtained at the second time. In some such embodiments generating a first spatial distribution of PPG pulse amplitudes based on the first set of light intensity values comprises generating an initial first spatial distribution of PPG amplitudes corresponding to the first time and a later first spatial distribution of PPG amplitudes corresponding to the second time. In some such embodiments generating a second spatial distribution of PPG pulse amplitudes based on the second set of light intensity values comprises generating an initial second spatial distribution of PPG amplitudes corresponding to the first time and a later second spatial distribution of PPG amplitudes corresponding to the second time. In some such embodiments comparing the first spatial distribution to the second spatial distribution comprises comparing the initial first spatial distribution to the initial second spatial distribution and comparing the later first spatial distribution to the later second spatial distribution.

In some such embodiments the method further comprises comparing the initial first spatial distribution to the later first spatial distribution and comparing the initial second spatial distribution to the later second spatial distribution. In such embodiments outputting a determination of whether the subject is experiencing an allergic reaction to the substance is additionally based on the comparing of the initial spatial distributions to the later spatial distributions.

In some embodiments the determination of whether the subject is experiencing an allergic reaction comprises a likelihood that the subject is experiencing an allergic reaction to the substance. In some embodiments the determination of whether the subject is experiencing an allergic reaction comprises an indication of the severity of an allergic reaction.

In some embodiments the skin region includes a first location at which a first substance has been applied and a second location at which a second substance has been applied. In some such embodiments outputting an indication of whether the subject is experiencing an allergic reaction to the substance comprises outputting a first indication of whether the subject is experiencing an allergic reaction to the first substance and a second indication of whether the subject is experiencing an allergic reaction to the second substance.

There is also provided, according to a second aspect of the invention, an apparatus for use in determining whether a subject is allergic to a substance. The apparatus comprises a processing unit arranged to perform the method of the first aspect.

Various other embodiments of the apparatus are also contemplated in which the processing unit is further configured to execute any of the above-described method steps.

There is also provided, according to a third aspect of the invention, a system for use in determining whether a subject is allergic to a substance. The system comprises an apparatus according to the second aspect; a light source arranged to emit at least one wavelength of visible light and at least one wavelength of infrared, IR, light; a camera, in communication with the apparatus, and a skin patch for maintaining the substance in contact with the skin of the subject. The camera is arranged to detect an intensity of the at least one wavelength of visible light and an intensity of the at least one wavelength of IR light. The camera is further arranged to output spatially distributed visible light intensity values and spatially distributed IR light intensity values. The skin patch comprises a material which is at least partially transparent to the at least one wavelength of visible light and at least partially transparent to the at least one wavelength of IR light.

In some embodiments the system further comprises a projector in communication with the apparatus. In some such embodiments the projector is arranged to project a mark onto the skin of the subject at a location selected by the apparatus.

In some embodiments the apparatus is arranged to cause the camera to output a continuous time-series of intensities of the at least one wavelength of visible light and to simultaneously output a continuous time-series of intensities of the at least one wavelength of IR light, during a monitoring period.

There is also provided, according to a fourth aspect of the invention, a skin patch for use in the system of the third aspect. The skin patch is arranged to maintain a substance in contact with the skin of a subject. The skin patch comprises a material which is at least partially transparent to the at least one wavelength of visible light and at least partially transparent to the at least one wavelength of IR light.

There is also provided, according to a fifth aspect of the invention, a computer program product, comprising computer readable code embodied therein, the computer readable code being configured such that, on execution by a suitable computer or processor, the computer or processor performs the method of the first aspect.

### BRIEF DESCRIPTION OF THE DRAWINGS

For a better understanding of the invention, and to show more clearly how it may be carried into effect, reference will now be made, by way of example only, to the accompanying drawings, in which:

FIG. 1a shows a prior art allergy test patch attached to a subject;

FIG. 1b shows the subject of FIG. 1a, after the test patch has been removed;

FIG. 2 shows examples of skin reactions to a patch test;

FIG. 3 shows an apparatus for determining whether a subject is allergic to a given substance, according to a first specific embodiment of the invention;

FIG. 4 is a flow chart showing a method for determining whether a subject is allergic to a given substance, according to a general embodiment of the invention;

FIG. 5 is a flow chart showing an analysis process, according to a general embodiment of the invention;

FIG. 6a is a photograph of a skin reaction to an allergy test; and

FIG. 6b is a PPG image of the skin reaction of FIG. 5a.

### DETAILED DESCRIPTION OF THE PREFERRED EMBODIMENTS

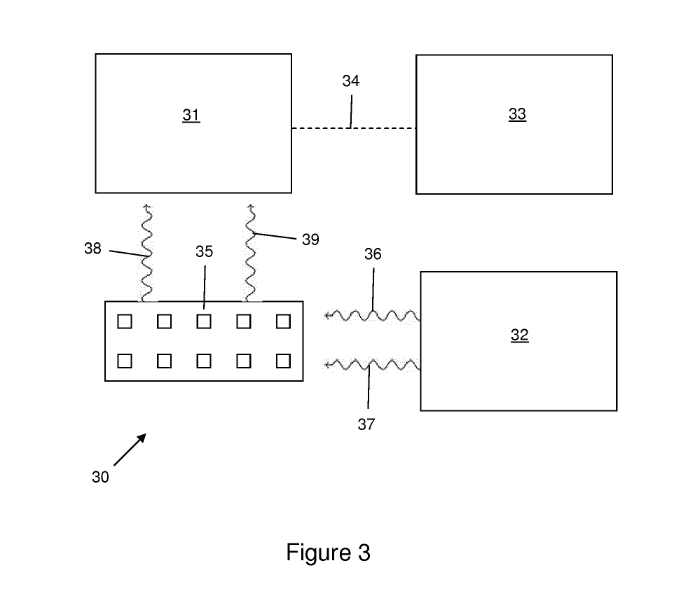

FIG. 3 shows an apparatus 30 for determining whether a subject is allergic to a substance, according to a first embodiment of the invention. The apparatus 30 comprises a camera 31, a light source 32, and a controller 33 in communication with the camera 31 via a communications link 34. In some embodiments a set of cameras is used instead of single camera 31. In some embodiments a set of light sources are used instead of a single light source 32. The apparatus 30 also comprises a skin patch 35 for maintaining at least one potentially allergenic substance in contact with a subject's skin.

The controller 33 controls the operation of the apparatus 30 and can be or can comprise one or more processors, processing units, multi-core processors or modules that are configured or programmed to control the apparatus 30 to determine whether a subject is allergic to a substance as described below.

The light source 32 is configured to emit light of at least two wavelengths, including at least one wavelength of visible light 36 and at least one wavelength of infrared (IR) 37. In preferred embodiments the light source 32 emits light over a broad range of wavelengths covering the visible and near IR spectrum. In some embodiments the light source 32 comprises a visible light source and a separate IR light source. In some embodiments the light source 32 emits visible light and IR light simultaneously, and in other embodiments the light source 32 emits visible light and IR light sequentially. The camera 31 can be provided with at least two filters corresponding to the wavelengths emitted by the light source 32 (i.e. at least one visible wavelength filter and at least one IR wavelength filter). The camera 31 can therefore detect reflected visible light 38 and reflected IR 39. In preferred embodiments the camera 31 is a charge-coupled device (CCD) camera with a high dynamic range. In some embodiments the camera 31 is configured to detect reflected visible light and reflected IR together so as to enable light intensity values to be detected at both wavelengths simultaneously. In other embodiments, multiple cameras 31 can be provided that each detect light intensity values at respective wavelengths (e.g. visible and IR), so as to enable light intensity values to be detected at both wavelengths simultaneously. In still other embodiments, the camera 31 can be configured to alternately or sequentially detect light at visible and IR wavelengths so as to obtain light intensity values at visible and IR wavelengths sequentially.

The skin patch 35 is at least partially transparent to the wavelengths emitted by the light source 32. It will be appreciated that the skin patch 35 needs to be sufficiently transparent for the light emitted by the light source 32 to reach the skin through the patch material, and also for the reflected light to be detectable by the camera 31 once it has passed through the patch material. Various suitable biocompatible and hypoallergenic materials are known in the art. The skin patch 35 is at least partially coated with an adhesive layer to enable attachment to the subject's skin. The communications link 34 is a two-way communications link which permits the controller 33 to send control signals to the camera 31 and to receive image data from the camera 31 (although it will be appreciated that in alternative embodiments a one-way communications link which only permits the sending of control signals from the controller 33 to the camera 31 could be used instead). The communications link 34 is a wireless communications link, although alternative embodiments are envisaged in which communications link 34 comprises a cable and/or circuitry.

In some embodiments the apparatus 30 further comprises a projector, to project images or marks onto a skin area of a subject, for example to assist in achieving optimal placement of the skin patch 34 on the subject's skin. In such embodiments the projector is connected to the controller 33 by a communications link which permits control signals to be sent from the controller 33 to the projector.

The apparatus 30 uses photoplethysmography (PPG) to detect and analyze a subject's skin properties. Conventional PPG is a simple and low-cost optical technique in which light at at least one visible wavelength is emitted into the skin region of interest, and the reflected light (or alternatively the transmitted light) at the emitted wavelength(s) is measured by a photodetector. The changing intensity of the reflected light corresponds to changes in the perfusion of the skin region of interest. Conventional PPG systems require the light source and photodetector to directly contact the skin, and therefore only allow the skin area directly below the sensor to be measured. Many conventional PPG systems emit light at two or more visible wavelengths, which improves the robustness of the measurements to noise, skin motion and changes in ambient light levels

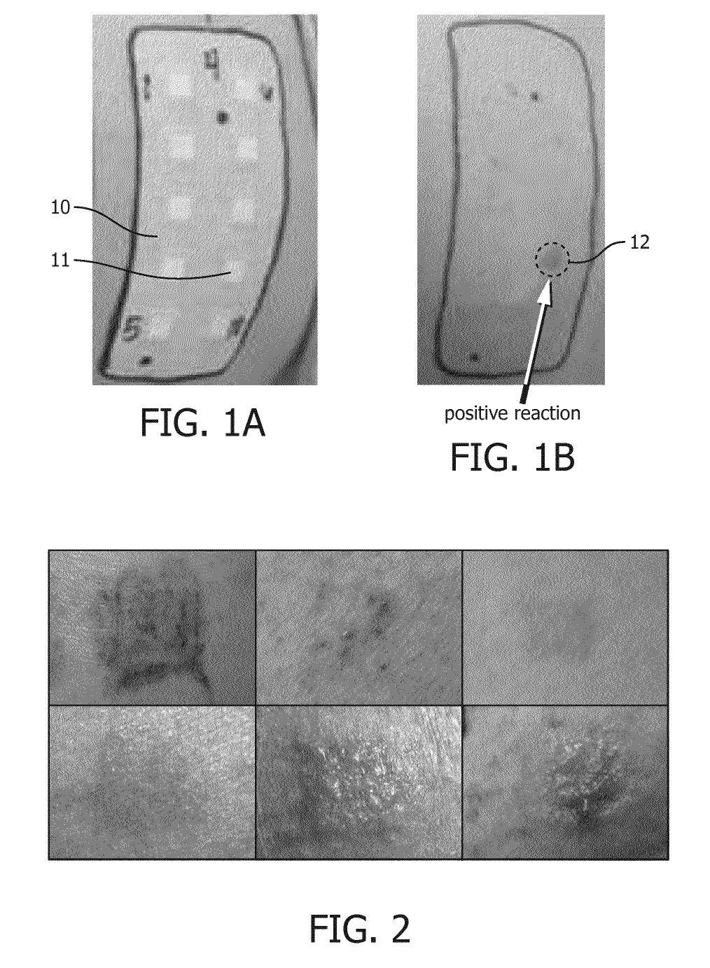

By contrast, embodiments of the invention use camera-based reflective mode PPG, in which the skin region of interest (i.e. the region including the allergy test patch 35) is filmed by a camera located at a distance from the skin's surface. The distance between the camera and the skin surface is sufficient that the entire skin region of interest (e.g. the entire area to which substances have been applied) is within the field of view of the camera. Furthermore, the skin region of interest is illuminated by IR as well as visible light (and the camera 31 detects reflections of IR as well as visible light). Either the detected light intensities in every single pixel, or the mean detected light intensity of a group of pixels, can be considered as PPG time signals. The PPG time signals represent the development of perfusion in the skin region of interest. In the embodiment of FIG. 1, the controller 33 is configured to analyze small groups (blocks) of pixels rather than individual pixels, so a PPG time signal is generated on the basis of the mean intensity of each group of pixels. The number of pixels in a block is selectable in dependence on the particular application. Larger blocks (i.e. containing more pixels) provide a better signal-to-noise ratio (SNR), but smaller blocks (i.e. containing fewer pixels) provide better spatial resolution. In alternative embodiments the controller 33 is configured to analyze each individual pixel. Because the apparatus 30 uses IR as well as visible light, separate PPG signals are generated on the basis of reflected IR light and on the basis of reflected visible light.

Each PPG time signal comprises a pulsatile (‘AC’) physiological waveform attributed to cardiac synchronous changes in the blood volume with each heart beat, and is superimposed on a slowly varying (‘DC’) baseline with various lower frequency components attributed to respiration, sympathetic nervous system activity and thermoregulation. The controller 33 processes the signals acquired by the camera 31 in a similar way to how PPG signals are processed in a conventional PPG system (such as a pulse oximeter), and thereby generates a spatial map of the pulse amplitude. The controller 33 processes the IR PPG signals separately from the visible light PPG signals; so that two separate spatial maps (i.e. an IR-based map and a visible light-based map) are generated.

The pulse amplitude (pulsatility) of the PPG signal is influenced by local skin properties. For instance, irritation of the skin due to an allergic reaction or a mechanical impact increases the amplitude of a PPG signal extracted from under the skin surface in the irritated region. Advantageously, such changes can be detected by a camera-based PPG system (such as the apparatus 30) before a reaction becomes visible on the surface of the skin. This means that embodiments of the invention can generate an allergy test result much more quickly than conventional skin patch testing methods. Furthermore, it enables real-time monitoring of the progression of a skin reaction.

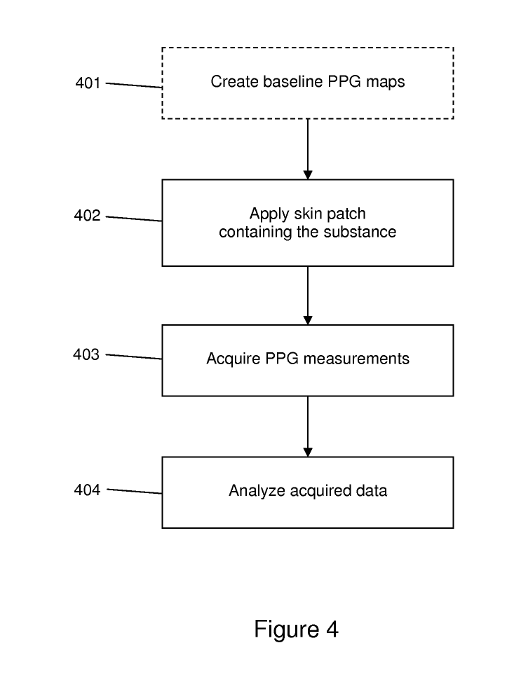

An exemplary method for determining whether a subject is allergic to a given substance will now be described with reference to FIG. 4. In the first step 401 baseline IR and visible PPG maps of a region of the subject's skin (i.e. a region in which it is desired to place the test patch 35) are created. As explained above, the creation of the baseline maps involves the controller 33 analyzing the PPG pulsatility for at least one visible wavelength and at least one IR wavelength, per block of pixels. The baseline maps each comprise a spatial distribution of PPG amplitude. Localized areas of higher PPG amplitude correspond to skin areas which already have local irritation, sunburns, scars, etc. In preferred embodiments the controller 33 is configured to automatically detect areas of pre-existing irritation, etc. (e.g. using any suitable image analysis techniques known in the art). Ideally, test patches should not be placed on areas which already exhibit irritation or another skin property which has the effect of increasing the amplitude of a PPG signal, because such pre-existing irritation can make it difficult to analyze and interpret a reaction caused by the test patch. However; this is not always possible (e.g. if a subject is suffering from a widespread skin condition or has extensive scarring). Embodiments of the invention are particularly advantageous in such situations, because the baseline PPG amplitudes can be subtracted from the PPG amplitudes obtained after the test patch has been applied, enabling an estimation to be made of how an existing skin condition is contributing to an observed reaction to the test patch.

It should be noted that the creation of baseline PPG maps is not essential to the invention (and thus step 401 is represented by a dashed box in FIG. 4). Embodiments of the invention are contemplated in which no baseline maps are created. Such embodiments are suitable for use on subject's who are known not to have any pre-existing skin conditions in the skin region where it is intended to apply the test patch. Omitting the baseline map creation step can advantageously reduce the time taken to perform the method and thus the inconvenience caused to the subject in such situations.

In step 402 the test patch 35 is attached to the subject's skin, e.g. by a medical professional. The baseline maps created in step 401 are used to select a location for the test patch 35 (In embodiments where no baseline maps are created, the expertise of a medical professional is relied upon in selecting the patch location). Ideally, the selected location does not include any areas having pre-existing properties (irritation, sunburn, etc.) which increase the PPG amplitude. In some embodiments in which the apparatus 30 further comprises a projector, the controller 33 causes the projector to project images or marks onto the patients skin to assist in attaching the test patch 35 at an optimal location. In some such embodiments the projector projects marks which highlight the location of regions having pre-existing PPG amplitude raising properties, so that the medical professional attaching the test patch 35 can easily avoid these regions. Alternatively or additionally, in such embodiments the projector can project an outline of the test patch at an optimal location. In some embodiments the optimal location is automatically determined by the controller 33 (e.g. using any suitable image analysis techniques known in the art).

In step 403, PPG measurements of the skin region underlying the test patch 35 are acquired, e.g. using the light source 32 and camera 31. In some embodiments the measurement is performed continuously during a given time period after (and optionally during and/or before) attachment of the patch (hereinafter referred to as the measurement period). In alternative embodiments a spot-check is performed at predetermined intervals. Preferably measurements are acquired to cover a time point 24 hours after attachment of the patch. Advantageously this enables the detection of crescendo or decrescendo scoring patterns, which are respectively suggestive of allergic and non-allergic reactions. In some embodiments measurements are acquired in accordance with the guidelines of the International Contact Dermatitis Research Group. In embodiments where continuous measuring is performed, the output of step 403 comprises a time series of spatially distributed light intensity values for visible light (i.e. one value for each pixel of the camera 31 per unit of time) and a time series of spatially distributed light intensity values for IR light. In embodiments where a series of spot-checks are performed, the output of this step comprises spatial distributions of light intensity values (visible and IR) for a single point in time. The number of such distributions produced will, of course, depend on the number of spot-checks performed.

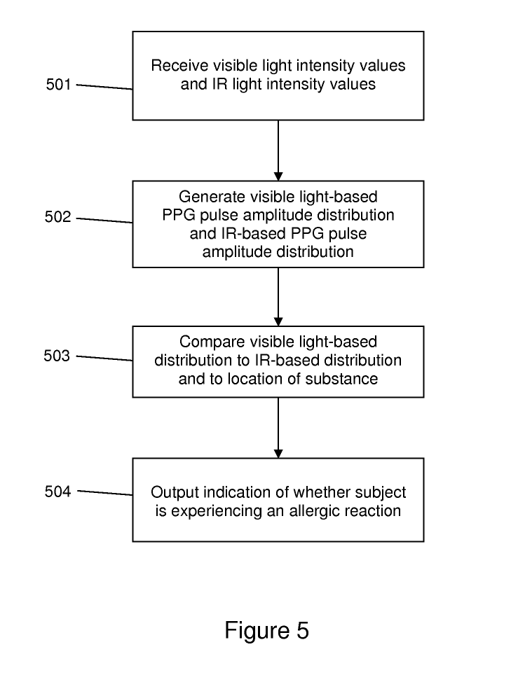

In step 404 the PPG data produced in step 403 is analyzed, e.g. by the controller 33. FIG. 5 illustrates the process used for the analysis. In a first step 501, light intensity values are received, e.g. by the controller 33 from the camera 31. The received light intensity values comprise a first set of spatially distributed visible light intensity values covering a skin region of the subject including the location at which the substance has been applied, and a second set of spatially distributed IR light intensity values covering the same skin region. As explained above, the camera 31 can acquire visible light intensity values and IR light intensity values simultaneously, so the visible light values and the IR values correspond to the same point(s) in time. Alternatively the camera 31 can acquire the visible light intensity values and IR light intensity values sequentially (e.g. alternating visible light values and IR light intensity values). Where the intensity values are acquired sequentially, since the pulsatility (amplitude) of a heart beat signal varies physiologically, the time between obtaining intensity values at each wavelength should be limited by, for example, inter-beat timing.

In step 502, a first (visible) spatial distribution of PPG pulse amplitudes is generated (e.g. by the controller 33) based on the received visible light intensity values. A second (IR) spatial distribution of PPG pulse amplitudes is also generated, based on the received IR light intensity values. In some embodiments a PPG pulse amplitude value is calculated for each pixel (i.e. for each light intensity value). In alternative embodiments a PPG pulse amplitude value is calculated for a block of pixels, using an average of the light intensity values of the pixels in the block. In some embodiments (e.g. embodiments in which the camera continuously monitors the skin region for the duration of the measurement period) the generated spatial distributions are time-varying, covering part or all of the measurement period. In other embodiments (e.g. embodiments in which one or more spot-checks are performed), the generated spatial distributions are static.

In embodiments in which baseline maps have been created, the processor 33 also receives the baseline maps (e.g. by retrieving them from a memory, or receiving them from a remote server). Each generated spatial distribution is then compared to the corresponding baseline map (i.e. the IR spatial distribution generated in step 502 is compared to the IR baseline map created in step 401, and the visible spatial distribution generated in step 502 is compared to the visible baseline map created in step 401). Where the generated spatial distributions are time-varying, each distribution may be compared to its corresponding baseline map for every time point of the measurement period, or at a predetermined number of time points distributed over the measurement period. The differences in PPG pulse amplitudes between each generated distribution and the corresponding baseline map are calculated for each pixel (or each block) (i.e. the contribution of the “baseline skin condition” is subtracted). For each generated distribution, a “difference map” is thereby created. The difference maps represent the reaction of the subject's skin to the test patch 35. They can therefore be considered to be corrected spatial distributions of PPG pulse amplitude. The corrected spatial distributions may be static (i.e. representing the skin reaction at a particular point in time) or time-varying, in a similar manner to the original (i.e. uncorrected) PPG amplitude distributions.

IR light penetrates more deeply into skin tissue than visible light. Therefore the PPG data acquired using IR light indicates the tissue perfusion at a greater depth below the skin surface than the PPG data acquired using visible light. It also means that a skin reaction which affects the PPG pulse amplitude will be detectable in the IR PPG data at an earlier time point than in the visible light PPG data. A more severe skin reaction will extend deeper beneath the skin's surface than a less severe reaction. Thus, by analysing the IR PPG maps and the visible light PPG maps of the reaction for various time points, the progression of the skin reaction at two different depths can be observed. This enables the severity of the reaction to be determined, and therefore the reaction to be classified as allergic (or otherwise), significantly more accurately than is possible using only visible light PPG data.

In step 503 the generated visible light spatial distribution of PPG pulse amplitudes is compared to the generated IR spatial distribution of PPG pulse amplitudes. In some embodiments this is done by comparing the pulse amplitudes of corresponding pixels (or blocks) in each spatial distribution. In some embodiments this is done by comparing the pixel values of corresponding pixels (or the average pixel values of corresponding blocks) in each spatial distribution. The pixel values of skin region experiencing a strong reaction are expected to differ from the pixel values of a skin region experiencing no reaction by approximately 10 points. In embodiments where corrected spatial distributions have been created, the corrected versions are used for this comparison. In some embodiments, for each pixel (or block) in a spatial distribution of PPG pulse amplitudes, the pulse amplitude for that pixel/block is compared to a set of predefined criteria to determine whether the skin region corresponding to the block is exhibiting a reaction. In preferred embodiments the same criteria are applied to the visible light spatial distribution of PPG pulse amplitudes and to the IR spatial distribution of PPG pulse amplitudes. This allows an objective comparison of the reaction at different depths to be made.

In some embodiments the set of predefined criteria comprises a set of predefined thresholds. In some embodiments the thresholds are defined in terms of pixel values. In some embodiments the set of predefined thresholds comprises a minimum pixel value such that pixels (or blocks) having a pixel value higher than the threshold are deemed to represent a skin area that this experiencing a reaction. In some such embodiments the set of predefined thresholds comprises a first minimum pixel value and a second minimum pixel value, such that pixels (or blocks) having a pixel value higher than the first minimum pixel value but less than or equal to the second minimum pixel value are deemed to represent a skin area that this experiencing a moderate reaction, and pixels (or blocks) having a pixel value higher than the second minimum pixel value are deemed to represent a skin area that this experiencing a strong reaction.

The PPG pulse amplitude distributions are also compared to the location at which the substance was applied. In preferred embodiments the PPG pulse amplitudes corresponding to skin regions where a substance has been applied are compared to PPG pulse amplitudes corresponding to skin regions where no substance has been applied. The regions where no substance has been applied thereby serve as a reference or a base line. This is advantageous because the overall PPG pulse amplitude of a person's skin might change due to various factors, including blood pressure, heart rate, body position, etc. Therefore, in order to detect changes in PPG pulse amplitude which are caused only by a skin reaction to a substance it is beneficial to compare the PPG pulse amplitudes from skin regions where it is known that a substance has been applied to PPG pulse amplitudes simultaneously acquired from skin regions where no substance has been applied. For example, if a change in PPG pulse amplitude is exhibited by a skin region where a substance has been applied between a first time point and a second time point, but a change of similar magnitude is also exhibited by a skin region where no substance has been applied between the same time points, this indicates that the change in the region where a substance has been applied is probably not caused by a reaction to that substance.

The results of the comparisons are then used to determine whether the subject is experiencing an allergic reaction to the substance. For example, if the comparisons to the predefined criteria indicate that the subject is exhibiting a reaction in a given region, and that region corresponds to the location of the substance, and the same reaction is not also exhibited in a region where no substance has been applied, in some embodiments it will consequently be determined that the subject is experiencing an allergic reaction to the substance. By contrast, in such embodiments if the predefined criteria indicate that the subject is exhibiting a reaction in a given region, and that region does not correspond to the location of the substance, it will be determined that the subject is not experiencing an allergic reaction to the substance (but they may, of course, be experiencing an allergic reaction to a different substance).

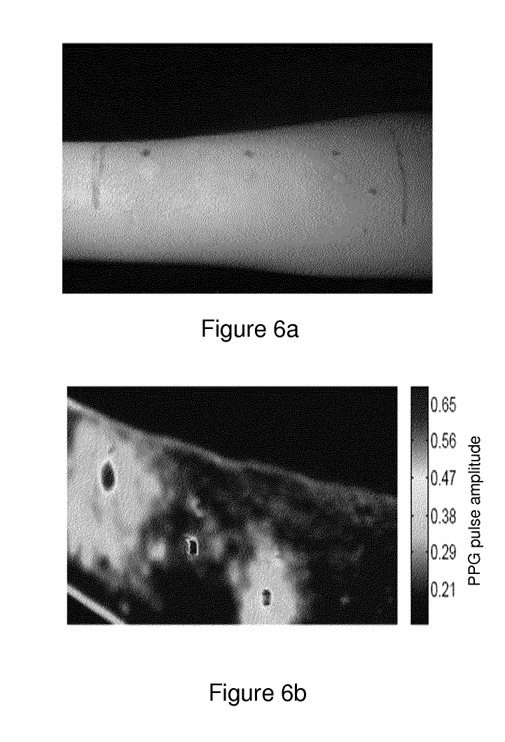

FIG. 6b shows an example of a visible light PPG map of a subject's forearm to which three potential allergens have been applied. Different PPG pulse amplitudes are represented by different colours, as shown by the scale on the right of this figure. It can clearly be seen from FIG. 6b that the subject is experiencing allergic reactions to the left-hand substance and the right-hand substance, but not the middle substance. It can also clearly be seen that the left-hand reaction is significantly more severe than the right-hand reaction. FIG. 6a is a photograph of the same forearm. It will be appreciated that it is much more difficult to accurately assess the differing severity of the three reactions from the photograph as compared to from the PPG amplitude map.

In step 504 a determination of whether or not the subject is experiencing an allergic reaction to the substance is output. In some such embodiments the output comprises an indication of which skin areas are considered to be exhibiting an allergic reaction. In some embodiments, if it is determined that the subject is experiencing an allergic reaction, the output further comprises an indication of a severity level of the allergic reaction. In some such embodiments, the indication of a severity level comprises a numerical score. In some embodiments the output comprises a likelihood value; indicative of how likely it is that a given area of skin is exhibiting an allergic reaction. In some such embodiments the likelihood value comprises a numerical score.

Although the embodiments of the invention in FIGS. 3 and 4 use camera-based PPG imaging together with a skin patch-based allergen application technique, it will be appreciated that the invention can be used together with other means of applying a potential allergen to a subject. For example, the skin patch 35 can be omitted from the apparatus 30, and the potential allergen substance(s) can instead be applied using prick-test techniques, intracutaneous injection, or any other substance application technique known in the field of allergy testing.

There is therefore provided a method and apparatus that allow a skin reaction to a substance to be evaluated quickly and objectively to determine whether a subject is experiencing an allergic reaction to the substance with high reliability.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method performed in a processing unit for use in determining whether a subject is allergic to a substance, the method comprising:
receiving a first set of spatially distributed light intensity values covering a skin region of the subject including a location at which the substance has been applied; wherein the light intensity values in the first set are intensities of visible light;
receiving a second set of spatially distributed light intensity values covering the skin region, wherein the light intensity values in the second set are intensities of infrared, IR, light;
generating a first spatial distribution of photoplethysmogram, PPG, pulse amplitudes based on the first set of light intensity values;
generating a second spatial distribution of PPG pulse amplitudes based on the second set of light intensity values;
comparing the first spatial distribution to the second spatial distribution, and, for each of the first and second spatial distributions, comparing PPG pulse amplitudes corresponding to the location at which the substance has been applied to PPG pulse amplitudes corresponding to a location at which the substance has not been applied; and
outputting an indication of whether the subject is experiencing an allergic reaction to the substance and the severity of the allergic reaction based on the results of the comparisons.

2. A method according to claim 1, wherein comparing the first spatial distribution to the second spatial distribution comprises:
identifying regions of high pulse amplitude in the first spatial distribution by comparing the pulse amplitudes in the first spatial distribution to a first threshold;
identifying regions of high pulse amplitude in the second spatial distribution by comparing the pulse amplitudes in the second spatial distribution to a second threshold; and
comparing the identified regions of high pulse amplitude in the first spatial distribution to the identified regions of high pulse amplitude in the second spatial distribution.

3. A method according to claim 1, further comprising:
receiving a first baseline set of spatially distributed light intensity values covering the skin region, wherein the light intensity values in the first baseline set are intensities of visible light;
receiving a second baseline set of spatially distributed light intensity values covering the skin region, wherein the light intensity values in the second baseline set are intensities of infrared, IR, light;
generating a first baseline spatial distribution of PPG pulse amplitudes based on the first baseline set of light intensity values; and
generating a second baseline spatial distribution of PPG pulse amplitudes based on the second baseline set of light intensity values;
wherein the light intensity values in the first and second baseline sets were obtained before the substance was applied to the skin region.

4. A method according to claim 3, further comprising:
subtracting the first baseline spatial distribution of PPG pulse amplitudes from the first spatial distribution of PPG pulse amplitudes to generate a corrected first spatial distribution of PPG pulse amplitudes;
subtracting the second baseline spatial distribution of PPG pulse amplitudes from the second spatial distribution of PPG pulse amplitudes to generate a corrected second spatial distribution of PPG pulse amplitudes;
wherein comparing the first spatial distribution to the second spatial distribution comprises comparing the corrected first spatial distribution to the corrected second spatial distribution, and wherein comparing PPG pulse amplitudes corresponding to the location at which the substance has been applied to PPG pulse amplitudes corresponding to a location at which the substance has not been applied comprises comparing corrected PPG pulse amplitudes corresponding to the location at which the substance has been applied to corrected PPG pulse amplitudes corresponding to the location at which the substance has not been applied.

5. A method according to claim 3, further comprising:
identifying regions of high pulse amplitude in the first baseline spatial distribution by comparing the pulse amplitudes in the first baseline spatial distribution to a threshold;
identifying regions of high pulse amplitude in the second baseline spatial distribution by comparing the pulse amplitudes in the second baseline spatial distribution to a threshold; and
selecting a skin location for application of the substance based on the identified regions of high pulse amplitude in the first and second baseline spatial distributions.

6. A method according to claim 5, wherein selecting a skin location for application of the substance comprises selecting a skin location which is not within any of the identified regions of high pulse amplitude, and/or wherein the method further comprises applying a mark to the subject's skin at the selected location.

7. A method according to claim 1, wherein:
the first set of spatially distributed light intensity values comprises spatially distributed light intensity values covering the skin region obtained at a first time, and spatially distributed light intensity values covering the skin region obtained at a second, later, time;
the second set of spatially distributed light intensity values comprises spatially distributed light intensity values covering the skin region obtained at the first time, and spatially distributed light intensity values covering the skin region obtained at the second time;
generating a first spatial distribution of PPG pulse amplitudes based on the first set of light intensity values comprises generating an initial first spatial distribution of PPG amplitudes corresponding to the first time and a later first spatial distribution of PPG amplitudes corresponding to the second time;
generating a second spatial distribution of PPG pulse amplitudes based on the second set of light intensity values comprises generating an initial second spatial distribution of PPG amplitudes corresponding to the first time and a later second spatial distribution of PPG amplitudes corresponding to the second time; and
comparing the first spatial distribution to the second spatial distribution comprises comparing the initial first spatial distribution to the initial second spatial distribution and comparing the later first spatial distribution to the later second spatial distribution.

8. A method according to claim 7, further comprising comparing the initial first spatial distribution to the later first spatial distribution and comparing the initial second spatial distribution to the later second spatial distribution, wherein outputting a determination of whether the subject is experiencing an allergic reaction to the substance is additionally based on the comparing of the initial spatial distributions to the later spatial distributions.

9. A method according to claim 1, wherein the determination of whether the subject is experiencing an allergic reaction comprises a likelihood that the subject is experiencing an allergic reaction to the substance and/or an indication of the severity of an allergic reaction.

10. A method according to claim 1, wherein the skin region includes a first location at which a first substance has been applied and a second location at which a second substance has been applied; and wherein outputting an indication of whether the subject is experiencing an allergic reaction to the substance comprises outputting a first indication of whether the subject is experiencing an allergic reaction to the first substance and a second indication of whether the subject is experiencing an allergic reaction to the second substance.

11. An apparatus for use in determining whether a subject is allergic to a substance, comprising:
a processing unit arranged to perform the method of claim 1.

12. A system for use in determining whether a subject is allergic to a substance, comprising:
an apparatus according to claim 11;
a light source arranged to emit at least one wavelength of visible light and at least one wavelength of infrared, IR, light;
a camera, in communication with the apparatus, and arranged to:
detect an intensity of the at least one wavelength of visible light and an intensity of the at least one wavelength of IR light; and
output spatially distributed visible light intensity values and spatially distributed IR light intensity values; and
a skin patch for maintaining the substance in contact with the skin of the subject; wherein the skin patch comprises a material which is at least partially transparent to the at least one wavelength of visible light and at least partially transparent to the at least one wavelength of IR light.

13. A system according to claim 12, further comprising a projector in communication with the apparatus, and arranged to project a mark onto the skin of the subject at a location selected by the apparatus.

14. (canceled)

15. A computer program product, comprising computer readable code embodied therein, the computer readable code being configured such that, on execution by a suitable computer or processor, the computer or processor performs the method described in claim 1.
